(19) United States
(12) Patent Application Publication
(10) Pub. No.: US 2014/0246744 A1
(43) Pub. Date: Sep. 4, 2014

(54) METHOD OF MANUFACTURING RADIATION DETECTOR AND RADIATION DETECTOR

(71) Applicants: Masatomo Kaino, Seika-cho (JP); Satoshi Tokuda, Kusatsu (JP); Toshinori Yoshimuta, Takatsuki-shi (JP); Hiroyuki Kishihara, Kizugawa-shi (JP); Akina Yoshimatsu, Osaka-shi (JP); Toshiyuki Sato, Kyoto-shi (JP); Shoji Kuwabara, Ibaraki-shi (JP)

(72) Inventors: Masatomo Kaino, Seika-cho (JP); Satoshi Tokuda, Kusatsu (JP); Toshinori Yoshimuta, Takatsuki-shi (JP); Hiroyuki Kishihara, Kizugawa-shi (JP); Akina Yoshimatsu, Osaka-shi (JP); Toshiyuki Sato, Kyoto-shi (JP); Shoji Kuwabara, Ibaraki-shi (JP)

(73) Assignee: SHIMADZU CORPORATION, Kyoto-shi, Kyoto (JP)

(21) Appl. No.: 14/009,210

(22) PCT Filed: Mar. 19, 2012
(86) PCT No.: PCT/JP2012/001894

§ 371 Date: Oct. 1, 2013

(30) Foreign Application Priority Data
Apr. 1, 2011 (JP) 2011-081785

(51) Int. Cl. H01L 31/18; H01L 31/0272

(52) U.S. Cl. CPC H01L 31/18; H01L 31/0272
USPC 257/431; 438/95

(57) ABSTRACT

A graphite substrate is accommodated into a chamber where vacuum drawing is performed via a pump. Thereafter, carbon is heated under vacuum, whereby impurities in the carbon are evaporated causing the carbon to be purified. The carbon in the graphite substrate is purified, achieving suppression of the impurities as donor/acceptor elements and also metallic elements in the semiconductor layer of 0.1 ppm or less, the impurities being contained in the carbon in the graphite substrate. As a result, occurrence of leak current or an abnormal leak point enables to be suppressed, and thus abnormal crystal growth in the semiconductor layer enables to be suppressed.

### TECHNICAL FIELD

This invention relates to a method of manufacturing a radiation detector and the radiation detector for use in the medical, industrial, nuclear and other fields.

### BACKGROUND ART

Various semiconductor materials, especially monocrystals of CdTe (cadmium telluride) or CdZnTe (cadmium zinc telluride), for a conventional high-sensitive radiation detector have been researched and developed, and a part of them has become commercial. The radiation detector of this type applies bias voltage to a semiconductor layer composed of CdTe or CdZnTe to fetch signals. Here, adopting a conductive graphite substrate as a support substrate achieves omission of common electrodes for voltage application electrodes. See, for example, Japanese Unexamined Patent Publications No. 2008-71961A and No. 2005-012049A.

### SUMMARY OF INVENTION

### Technical Problem

On the other hand, when the semiconductor layer composed of the above CdTe or CdZnTe contains impurities, resistance decreases to increase leak current or generate an abnormal leak point. In addition, crystals in the semiconductor layer may be grown abnormally.

This invention has been made regarding the state of the art noted above, and its object is to provide a method of manufacturing a radiation detector and the radiation detector allowing suppression of occurrence of leak current or an abnormal leak point and thereby suppression of abnormal growth of crystals in a semiconductor layer.

### Solution to Problem

To overcome the above problems, Inventors have made intensive research and attained the following findings.

Specifically, in order to overcome the problems, impurities in the semiconductor layer have conventionally been decreased so as to suppress impurities as donor/acceptor elements in the semiconductor layer, the semiconductor layer being doped with the donor/acceptor elements. On the other hand, a graphite substrate is formed based on artificial or natural graphite (black lead). Accordingly, when no purification treatment is performed, no treatment is performed to the graphite substrate even containing impurities, such as Al, B, Ca, Cr, Cu, Fe, K, Mg, Mn, Na, Ni, Si, Ti, and V, to a detectable extent. Although a blocking layer is disposed between the graphite substrate and the semiconductor layer or the semiconductor layer is directly laminated on the graphite substrate to decrease the impurities in the semiconductor layer a portion of the semiconductor layer adjacent to the graphite substrate may be doped with the impurities. Such finding has been obtained.

This invention based on the above finding adopts the following configuration. One embodiment of the invention discloses a method of manufacturing a radiation detector. The radiation detector includes a semiconductor layer composed of CdTe (cadmium telluride) or CdZnTe (cadmium zinc telluride) and a graphite substrate for voltage application electrodes. The semiconductor layer converts radiation information to charge information upon incidence of radiation. The graphite substrate also serves as a support substrate and applies bias voltage to the semiconductor layer. The method includes purifying carbon as a primary element of the graphite substrate.

According to the method of manufacturing the radiation detector in the embodiment of the invention, the carbon in the graphite substrate is purified, achieving suppression of impurities as donor/acceptor elements and also a metallic element in the carbon of the graphite substrate. As a result, impurities (the donor/acceptor elements or the metallic element) dispersed into the semiconductor layer from the graphite substrate enables to be suppressed. Consequently, occurrence of leak current or an abnormal leak point due to the donor/acceptor elements with which the semiconductor layer is doped enables to be suppressed. Moreover, abnormal growth of crystals in the semiconductor layer enables to be suppressed, the abnormal growth caused from the metallic element with which the semiconductor layer is doped.

Examples of purifying the carbon include purifying carbon by heating the carbon. In this example, impurities in the graphite substrate enable to be removed with heating. Examples of heating the carbon also include heating carbon under vacuum causing impurities in the carbon to be evaporated for purifying the carbon. Examples of heating the carbon further include heating the carbon with gas supplied causing the carbon to be purified.

Examples of purifying the carbon also include cleaning the carbon. In this example, cleaning enables to eliminate impurities on a surface of the graphite substrate. Here, combination of both examples of heating the carbon and cleaning the carbon may be made.

Another embodiment of this invention discloses a radiation detector. The radiation detector includes a semiconductor layer composed of CdTe (cadmium telluride) or CdZnTe (cadmium zinc telluride), and a graphite substrate for voltage application electrodes. The semiconductor layer converts radiation information into charge information upon incidence of radiation. The graphite substrate also serving as a support substrate applies bias voltage to the semiconductor layer. The graphite substrate contains carbon with impurities as donor/acceptor elements in the semiconductor layer of 0.1 ppm or less.

In the method of manufacturing the radiation detector according to the embodiment, the carbon in the graphite substrate is purified, achieving the radiation detector having impurities as the donor/acceptor elements in the semiconductor layer of 0.1 ppm or less, the impurities being contained in the carbon in the graphite substrate. Consequently, occurrence of the leak current or the abnormal leak point enables to be suppressed.

In the radiation detector according to the embodiment, the impurities as the metallic element in the carbon are preferably of 0.1 ppm or less. The semiconductor layer is doped with the metallic element, crystal nuclei are generated, possibly leading to abnormal growth of crystals in the semiconductor layer. Then, the carbon in the graphite substrate is purified. Consequently, the radiation detector enables to be achieved also having the impurities as the metallic element of 0.1 ppm or less in the carbon in the graphite substrate. As a result, the abnormal growth of the crystals enables to be suppressed in the semiconductor layer.

### Advantageous Effects of Invention

According to the method of manufacturing the radiation detector of the embodiment, the carbon in the graphite substrate is purified. This enables to suppress occurrence of the leak current or the abnormal leak point. Moreover, the abnormal growth of the crystals enables to be suppressed in the semiconductor layer.

According to the method of manufacturing the radiation detector according to the embodiment, the carbon in the graphite substrate is purified, achieving the radiation detector having impurities as the donor/acceptor elements in the semiconductor layer of 0.1 ppm or less, the impurities being contained in the carbon of the graphite substrate. Moreover, the radiation detector enables to be achieved also having the impurities as the metallic element of 0.1 ppm or less, the carbon in the graphite substrate containing the impurities.

### BRIEF DESCRIPTION OF DRAWINGS

FIG. 1 is a longitudinal sectional view of a portion of a radiation detector adjacent to a graphite substrate according to one embodiment of this invention.

FIG. 2 is a longitudinal sectional view of a portion of the radiation detector adjacent to a read-out substrate according to the embodiment of this invention.

FIG. 3 is a circuit diagram illustrating the read-out substrate and a peripheral circuit.

FIG. 4 is a longitudinal sectional view in combination of the graphite substrate and the read-out substrate according to the embodiment of this invention.

FIG. 5 is a schematic view when heating the graphite substrate composed of carbon under vacuum.

FIG. 6 is a schematic view when heating the graphite substrate composed of the carbon with gas supplied.

### DESCRIPTION OF EMBODIMENTS

Description will be given of the embodiment of this invention hereinunder in detail with reference to the drawings.

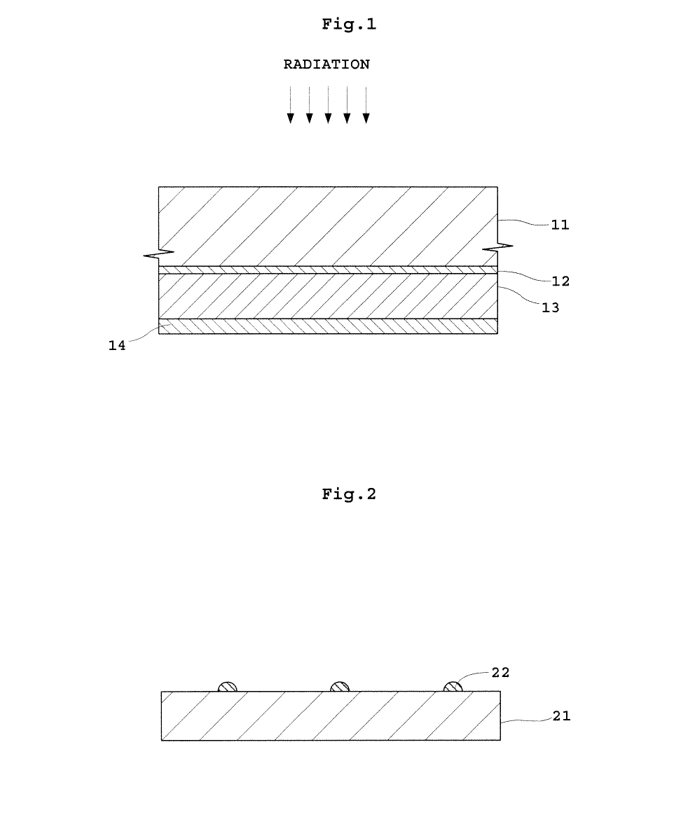

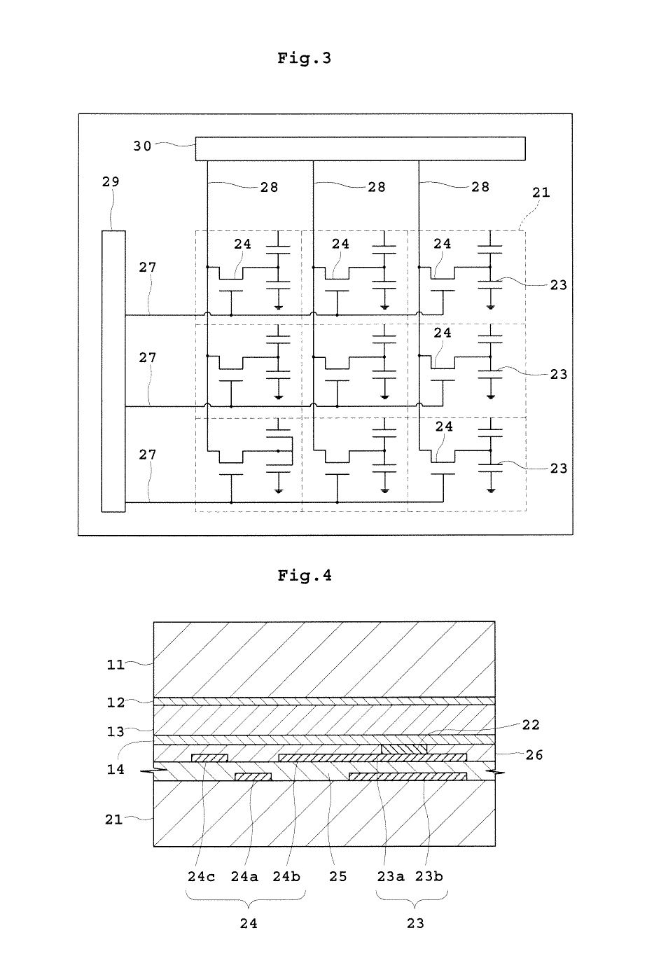

FIG. 1 is a longitudinal sectional view of a portion of a radiation detector adjacent to a graphite substrate according to one embodiment of this invention. FIG. 2 is a longitudinal sectional view of a portion of the radiation detector adjacent to a read-out substrate. FIG. 3 is a circuit diagram illustrating the read-out substrate and a peripheral circuit. FIG. 4 is a longitudinal sectional view in combination of the graphite substrate and the read-out substrate according to the embodiment.

As illustrated in FIGS. 1 to 4, a radiation detector is divided roughly into a graphite substrate 11 and a read-out substrates 21. As illustrated in FIGS. 1 and 4, an electron blocking layer 12, a semiconductor layer 13, and a hole blocking layer 14 are laminated in this order on the graphite substrate 11. As illustrated in FIGS. 2 and 4, the read-out substrate 21 includes pixel electrodes 22, which are to be mentioned later, and forms a pattern of capacitors 23, thin-film transistors 24, and the like. Here, FIG. 2 only illustrates the substrate 21 and the pixel electrodes 22. The graphite substrate 11 corresponds to the graphite substrate in this invention. The semiconductor layer 13 corresponds to the semiconductor layer in this invention.

As illustrated in FIG. 1, the graphite substrate 11 also serves as a support substrate and a voltage application electrode. In other words, the radiation detector is formed by the graphite substrate 11 for voltage application electrode, the graphite substrate applying bias voltage (i.e., bias voltage of −0.1 V/μm to 1 V/μm in this embodiment) to the semiconductor layer 13 and also serving as a support substrate. The graphite substrate 11 is composed of a plate made of conductive carbon graphite. The graphite substrate 11 adopts a planar plate (with a thickness of approximately 2 mm) having controlled baking conditions so as to conform to a coefficient of thermal expansion of the semiconductor layer 13.

The semiconductor layer 13 converts information of radiation (e.g., X-rays) into information of charge (carriers) upon incidence of the radiation. A polycrystalline-film composed of CdTe (cadmium telluride) or CdZnTe (cadmium zinc telluride) is used for the semiconductor layer 13. Here, the semiconductor layer 13 adopts coefficients of thermal expansion of CdTe of approximately 5 ppm/deg and that of CdZnTe varying in accordance with a Zn concentration.

A P-type semiconductor with ZnTe, Sb₂S₃, and Sb₂Te₃, for example, is used for the electron blocking layer 12. An N-type semiconductor or an ultra-high resistance semiconductor with CdS, ZnS, ZnO, and Sb₂S₃, for example, is used for the hole blocking layer 14. Here in FIGS. 1 and 4, the hole blocking layer 14 is formed continuously. Alternatively, the hole blocking layer 14 may be divided corresponding to the pixel electrodes 22 when it has a lower film resistor. When the hole blocking layer 14 is divided corresponding to the pixel electrodes 22, the divided hole blocking layer 14 each needs to be aligned with each pixel electrode 22 upon joining the graphite substrate 11 to the read-out substrate 21. Moreover, when the radiation detector has no problem on its properties, either the electron blocking layer 12 or the hole blocking layer 14 or both of them may be omitted.

As illustrated in FIG. 2, the pixel electrode 22 is formed on the read-out substrate 21 at a portion (pixel regions) corresponding to the capacity electrode 23a of the capacitor 23 (see FIG. 4), to be mentioned later, in the portion the pixel electrode 22 being bump-connected to the graphite substrate 11 via a conductive material (e.g., a conductive paste, an anisotropic conductive film (ACF), anisotropic conductive paste). As noted above, the pixel electrode 22 is formed for every pixel, and reads out the carriers converted in the semiconductor layer 13. A glass substrate is used for the read-out substrate 21.

As illustrated in FIG. 3, the read-out substrate 21 includes the capacitors 23 in the form of a charge storage capacitor and the thin-film transistors 24 in the form of a switching element being divided for every pixel to form a pattern. FIG. 3 merely illustrates the read-out substrate 21 with 3×3 pixels. In actual, the read-out substrate 21 is used having a size (e.g., 1,024×1,024 pixels) corresponding to the pixel number of a two-dimensional radiation detector.

As illustrated in FIG. 4, the capacity electrode 23a of the capacitor 23 and the gate electrode 24a of the thin-film transistor 24 are laminated over the read-out substrate 21 so as to cover an insulating layer 25. The reference electrode 23b of the capacitor 23 is laminated on the insulating layer 25 so as to face to the capacity electrode 23a via the insulating layer 25. A source electrode 24b and a drain electrode 24c of the thin-film transistor 24 except for a portion of connecting to the pixel electrode 22 are covered with an insulating layer 26. Here, the capacity electrode 23a and the source electrode 24b are electrically connected to each other. As illustrated in FIG. 4, the capacity electrode 23a and the source electrode 24b may be integrated simultaneously. The reference electrode 23b is grounded. The insulating layers 25, 26 are for example plasma SiN layers.

As illustrated in FIG. 3, a gate line 27 is electrically connected to a gate electrode 24a of the thin-film transistor 24 in FIG. 4, whereas a data line 28 is electrically connected to a drain electrode 24c of the thin-film transistor 24 in FIG. 4. The gate line 27 extends in a row direction of each pixel, whereas the data line 28 extends in a column direction of each pixel. The gate line 27 is orthogonal to the data line 28. The capacitor 23, the thin-film transistor 24, and the insulating layers 25, 26 in addition to the gate lines 27 and the data lines 28 are formed by pattern on a surface of the read-out substrate 21, composed of a glass substrate, using semiconductor thin-film fabrication techniques or fine processing techniques.

Moreover, as illustrated in FIG. 3, a gate drive circuit 29 and a read-out circuit 30 are also arranged around the read-out substrate 21. The gate drive circuit 29 is electrically connected to each gate line 27 extending in the row direction, and drives a pixel on each line in turn. The read-out circuit 30 is electrically connected to each data line 28 extending in the column direction, and reads out carriers of each pixel via the data line 28. The gate drive circuit 29 and the read-out circuit 30 are formed by a semiconductor integrated circuit made from silicone, for example, and electrically connect the gate lines 27 and the data lines 28, respectively, via an anisotropic conductive film (ACF).

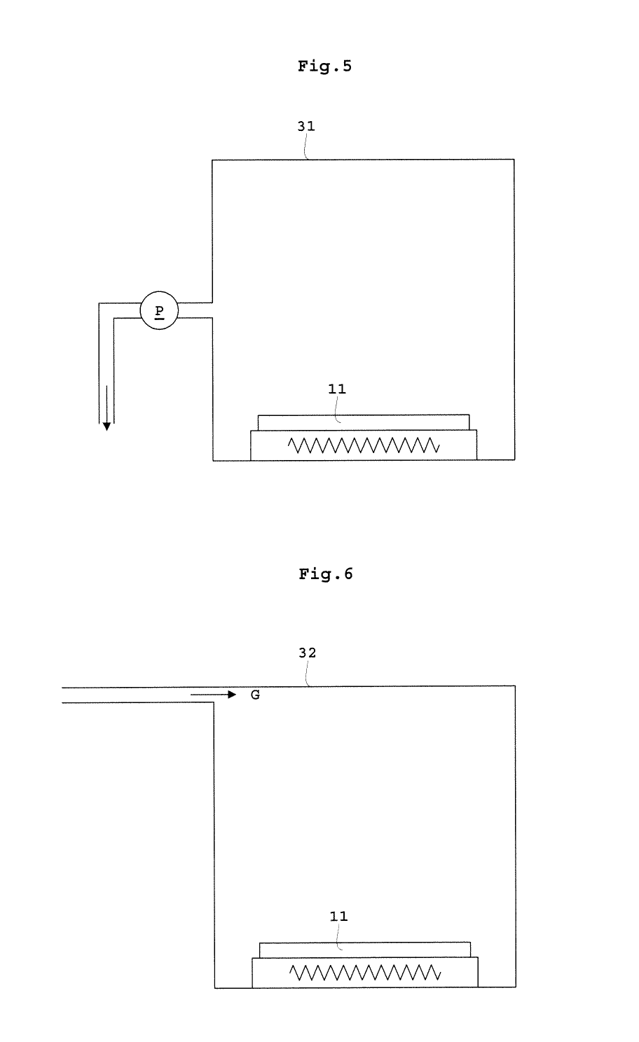

Description will be given next in detail of a method of manufacturing the radiation detector. FIG. 5 is a schematic view when heating the graphite substrate composed of carbon under vacuum. FIG. 6 is a schematic view when heating the graphite substrate composed of the carbon with gas supplied.

The graphite substrate 11 of relatively low prices and readily available is manufactured based on artificial or natural graphite (black lead), and thus contains various impurities. When the donor/acceptor elements as the impurities in the graphite substrate 11 relative to CdTe or CdZnTe are mixed into a CdZnTe film or a CdTe film due to thermo diffusion during film formation of the semiconductor layer 13, the donor/acceptor elements exert influences on film properties. The following elements have been known as the donor/acceptor elements relative to CdTe or CdZnTe.

A donor of Cd site: aluminum (Al), gallium (Ga), indium (In)

An acceptor of Cd site: lithium (Li), sodium (Na), copper (Cu), silver (Ag), gold (Au)

A donor of Te site: fluorine (F), chlorine (Cl), bromine (Br), iodine (I)

An acceptor of Te site: nitrogen (N), phosphorus (P), arsenic (As), antimony (Sb)

(Literature on donor and acceptor: Acceptor states in CdTe and comparison with ZnTe. E. molva et al. 1984, Shallow donoes in CdTe. L. M. Francou et al. 1990, etc.).

These elements generates excess electrons or positive holes relative to a CdTe or CdZnTe-based group II-VI compound semiconductor film, and thus mixing a trace quantity of these elements causes a film with lower resistance. Mixing of these elements also leads to unintended formation of pn junction, causing abnormal current-voltage properties. According to various literatures, CdTe and CdZnTe is significantly made p-type or n-type at an impurity concentration of 10¹⁵ cm⁻³ or more.

From these results, leak current increases entirely, or an abnormal leak point is generated where leak current is extremely high partially. Consequently, a signal-to-noise ratio of the radiation detector decreases, or image defects occur when the radiation detector is applied to an image.

Other than the donor/acceptor elements, an element such as magnesium (Mg), calcium (Ca), iron (Fe), Co (cobalt), nickel (Ni), and titanium (Ti) is a relatively common metallic element, and thus the element may be mixed into the graphite substrate 11. The metallic element mixed from the graphite substrate 11 into the CdTe film or the CdZnTe film constitutes crystal nuclei during crystal growth in film formation. This causes abnormal growth of crystals, and thus avoids homogenization of film properties.

Accordingly, in order to avoid the influences noted above, the carbon in the graphite substrate 11 is purified such that the graphite substrate 11 is controlled to have impurities of the above on a surface and inside thereof of 0.1 ppm or less. For purification of the carbon in the graphite substrate 11, the carbon is heated by an approach illustrated in FIG. 5 or 6.

In FIG. 5, the graphite substrate 11 is accommodated into a chamber 31 where vacuum drawing is performed via a pump P. Thereafter, carbon is heated under vacuum, whereby impurities in the carbon are evaporated causing the carbon to be purified. Here, a heating temperature is approximately 1000° C.

In FIG. 6, the graphite substrate 11 is accommodated into a chamber 32 where gas G is supplied. The gas G is preferably inert gas unreactive to the graphite substrate 11, and rare gas (He, Ne, Ar) or nitrogen (N₂) is used for the gas G. Then the carbon is heated with the gas G supplied to be purified. Here, a heating temperature is 2000° C. or more.

The carbon is heated with the approach illustrated in FIG. 5 or 6 to be purified. The purification causes various impurities on the surface or inside of the graphite substrate 11 to be of 0.1 ppm or less. Here, a threshold is set to be 0.1 ppm or less. The threshold represents measuring limit or less measured by a microanalysis method such as an inductive plasma atomic emission spectrometry, an atomic absorption method, an absorptiometric method, and a secondary ion composition analysis method. The approach illustrated in FIG. 5 or 6 achieves suppression of impurities to be of 0.1 ppm or less, which is the measuring limit or less.

Thereafter, the electron blocking layer 12 is laminated on the purified graphite substrate 11 by a sublimation method, an evaporation method, a sputtering method, a chemical deposition method, an electro deposition method, or the like.

The semiconductor layer 13 in the form of a conversion layer is laminated on the electron blocking layer 12 by a sublimation method. In Example 1 of this invention, since an X-ray detector having energy of several tens keV to several hundreds keV is used, a CdZnTe film containing several mol % to several tens mol % of zinc (Zn) with a thickness of approximately 300 μm is formed as the semiconductor layer 13 by a proximity sublimation method. Of course, a CdTe film containing no element Zn may be formed as the semiconductor layer 13. Moreover, the semiconductor layer 13 may be formed by not only the sublimation method but also an MOCVD method. Alternatively, a polycrystalline-film semiconductor layer 13 of CdTe or CdZnTe may be formed through application of a paste containing CdTe or CdZnTe. Then planarization is performed to the semiconductor layer 13 by polishing or sandblasting processing in which blasting abrasive such as sand is conducted.

Thereafter, the hole blocking layer 14 is laminated on the planarized semiconductor layer 13 by a sublimation method, an evaporation method, a spattering method, a chemical deposition method, an electro deposition method, or the like.

Thereafter, as illustrated in FIG. 4, the graphite substrate 11 with the semiconductor layer 13 laminated thereon and the read-out substrate 21 are joined such that the semiconductor layer 13 and the pixel electrodes 22 are joined inside. As noted above, bump-connection is performed to a portion of the capacity electrode 23a not covered with the insulating layer 26 via a conductive material (e.g., a conductive paste, an anisotropic conductive film (ACF), an anisotropic conductive paste), whereby the pixel electrode 22 is formed on the portion via which the graphite substrate 11 is joined to the read-out substrate 21.

According to the method of manufacturing the radiation detector with the above construction, the carbon in the graphite substrate 11 is purified, achieving suppression of impurities as the donor/acceptor elements and also metallic elements in the semiconductor layer 13 contained in the carbon in the graphite substrate 11. Consequently, impurities (the donor/acceptor elements or the metallic elements) dispersed into the semiconductor layer 13 from the graphite substrate 11 enables to be suppressed. As a result, occurrence of leak current or an abnormal leak point due to the donor/acceptor elements with which the semiconductor layer 13 is doped enables to be suppressed. This achieves suppression in abnormal crystal growth in the semiconductor layer 13 caused from the metallic elements with which the semiconductor layer 13 is doped.

In the embodiment of this invention, the carbon is purified through heating. In the embodiment, the impurities in the graphite substrate 11 enable to be removed through heating. Examples of the heating include heating the carbon under vacuum as in FIG. 5 to cause the impurities in the carbon to be evaporated for purifying the carbon. Examples of the heating also include heating the carbon with the gas G supplied as in FIG. 6 for purifying the carbon.

According to the method of manufacturing the radiation detector in the embodiment, the carbon in the graphite substrate 11 is purified, achieving the radiation detector having the impurities as the donor/acceptor elements in the semiconductor layer 13 that the graphite substrate 11 contains of 0.1 ppm or less. As a result, occurrence of leak current or an abnormal leak point enables to be suppressed.

In the embodiment of this invention, the impurities as the metallic elements in the carbon are preferably of 0.1 ppm or less. When the semiconductor layer 13 is doped with the metallic elements, crystal nuclei are generated, which may lead to abnormal crystal growth in the semiconductor layer 13. Then, the carbon in the graphite substrate 11 is purified, achieving a radiation detector also having the impurities as the metallic elements in the carbon in the graphite substrate 11 of 0.1 ppm or less. As a result, suppression of abnormal crystal growth in the semiconductor layer 13 enables to be obtained.

This invention is not limited to the foregoing embodiment, but may be modified as follows:

(1) The foregoing embodiment has been described taking X-rays as an example of radiation. However, examples of radiation other than X-rays include gamma-rays and light, and thus radiation is not particularly limited.

(2) In the foregoing embodiment, the carbon is purified through heating. Alternatively, the impurities on the surface of the graphite substrate may be removed through cleaning. In addition, combination of the embodiment of heating the carbon and the modification of cleaning the carbon may be adopted.

### REFERENCE SIGNS LIST

11 . . . graphite substrate
13 . . . semiconductor layer
G . . . gas

1. A method of manufacturing a radiation detector with a semiconductor layer composed of CdTe (cadmium telluride) or CdZnTe (cadmium zinc telluride) and a graphite substrate for voltage application electrodes, the semiconductor layer converting radiation information to charge information upon incidence of radiation, the graphite substrate also serving as a support substrate and applying bias voltage to the semiconductor layer, the method comprising:
purifying carbon as a primary element of the graphite substrate.

2. The method of manufacturing the radiation detector according to claim 1, wherein
the purifying carbon is performed by heating the carbon.

3. The method of manufacturing the radiation detector according to claim 2, wherein
the purifying the carbon is performed by heating the carbon under vacuum causing impurities in the carbon to be evaporated.

4. The method of manufacturing the radiation detector according to claim 2, wherein
the purifying the carbon is performed by heating the carbon with gas supplied.

5. The method of manufacturing the radiation detector according to claim 1, wherein
the purifying the carbon is performed by cleaning the carbon.

6. The method of manufacturing the radiation detector according to claim 1, wherein
the purifying the carbon is performed by heating the carbon and cleaning the carbon.

7. The method of manufacturing the radiation detector according to claim 6, wherein
the purifying the carbon is performed by heating the carbon under vacuum causing impurities in the carbon to be evaporated.

8. The method of manufacturing the radiation detector according to claim 6, wherein
the purifying the carbon is performed by heating the carbon with gas supplied.

9. A radiation detector comprising:
a semiconductor layer composed of CdTe (cadmium telluride) or CdZnTe (cadmium zinc telluride) and converting radiation information into charge information upon incidence of radiation; and
a graphite substrate for voltage application electrodes also serving as a support substrate applies bias voltage to the semiconductor layer,
the graphite substrate containing carbon with impurities as donor/acceptor elements in the semiconductor layer of 0.1 ppm or less.

10. The radiation detector according to claim 9, wherein
a donor of Cd (cadmium) site is aluminum (Al), gallium (Ga), or indium (In), and
the aluminum (Al), the gallium (Ga), or the indium (In) is of 0.1 ppm or less.

11. The radiation detector according to claim 9, wherein
an acceptor of Cd (cadmium) site is lithium (Li), sodium (Na), copper (Cu), silver (Ag), or gold (Au), and
the lithium (Li), the sodium (Na), the copper (Cu), the silver (Ag), or the gold (Au) is of 0.1 ppm or less.

12. The radiation detector according to claim 9, wherein
a donor of Te (telluride) site is fluorine (F), chlorine (Cl), bromine (Br), or iodine (I), and
the fluorine (F), the chlorine (Cl), the bromine (Br), or the iodine (I) is of 0.1 ppm or less.

13. The radiation detector according to claim 9, wherein
an acceptor of Te (telluride) site is nitrogen (N), phosphorus (P), arsenic (As), or antimony (Sb), and
the nitrogen (N), the phosphorus (P), the arsenic (As), or the antimony (Sb) is of 0.1 ppm or less.

14. The radiation detector according to claim 9, wherein
the impurities as the metallic element in the carbon are of 0.1 ppm or less.

15. The radiation detector according to claim 14, wherein
the metallic element is magnesium (Mg), calcium (Ca), iron (Fe), cobalt (Co), nickel (Ni), and titanium (Ti), and
the magnesium (Mg), the calcium (Ca), the iron (Fe), the Co (cobalt), the nickel (Ni), and the titanium (Ti) is of 0.1 ppm or less.
